(19) United States
(12) Patent Application Publication
(10) Pub. No.: US 2015/0018927 A1
(43) Pub. Date: Jan. 15, 2015

(54) DEVICE FOR NEUROSTIMULATION

(71) Applicant: EBS TECHNOLOGIES GMBH, Kleinmachnow (DE)

(72) Inventor: Udo Warschewske, Berlin (DE)

(73) Assignee: EBS TECHNOLOGIES GMBH, Kleinmachnow (DE)

(21) Appl. No.: 14/370,700

(22) PCT Filed: Jan. 10, 2013
(86) PCT No.: PCT/EP2013/050335

§ 371 Date: Jul. 3, 2014

(30) Foreign Application Priority Data
Jan. 19, 2012 (DE) 202012000492.1

(51) Int. Cl. A61N 1/04

(52) U.S. Cl. CPC A61N 1/0472; A61N 1/0456
USPC 607/141

(57) ABSTRACT

The invention relates to a device for neurostimulation, comprising a mask, eyeglasses or similar eye attachment covering the eye region of the subject, which is designed to be optically sealed from the surroundings and has a lighting surface directed to the eye side which is connected to a source emitting light in the visible spectral range or contains such a light source, and having means for fastening to the head of the subject. According to the invention, there is a base element, open on one side, which receives the respective lighting surface and light source on the inside thereof, wherein there are electrode receptacles or electrode support surfaces on the base element above and/or below the region covering the eye region, furthermore a flexible, replaceable cushion-like or mat-like flat element can be fastened on the base element, which flat element has openings that are complementary to the electrode receptacles or electrode support surfaces, wherein the flat element comes in contact with the forehead area of the eye region of the subject when the device is used and the electrodes are designed to be epicutaneous.

The invention relates to a device for neurostimulation, comprising a mask, eyeglasses or similar eye attachment covering the eye area of the subject which is designed to be optically sealed from the surroundings and has an illuminated surface which is directed to the eye side and is connected to a source emitting light in the visible spectral range or contains such a light source, and having means for fixing it to the head of the subject, according to claim 1.

A device for the light stimulation of biological tissue, in particular for the eye area, is already known from DE 20 2011 050 692 U1. This device comprises at least one means generating a movable light spot and, furthermore, presetting a respiratory frequency for the user. Additionally, further means are provided which make light waves of the near infrared spectrum act upon the user's tissue.

The prior solution is intended to allow a stimulation of a subject on the basis of the improvement of the cardiovascular condition and, furthermore, of the state of health of the eye in association with strengthening the eye muscles.

The necessary illuminants of the device are each assigned to one or both eyes, and light sources for the near infrared spectral range are integrated in the device in the glabella region and/or temporal region and/or occipital region. The radiation angle with regard to the emitted infrared radiation should be very small so as to achieve a great depth of penetration.

In practice, the device is designed in the form of light glasses, i.e. in the manner of a frame or display. A position for another illuminant or a group of other illuminants is located in the middle of the display. A diffusing disc is provided in front of the illuminants. A plurality of first illuminants is distributed around the diffusing disc. The illuminants are controlled by a microcontroller. Preferably, the illuminants produce a movement pattern, e.g. in the form of the impression of a circle or a loop of the moving light spot.

The learning and relaxing device for the stimulation of the central nervous system and the brain waves of a human being according to DE 38 23 402 A1 involves the transmission of pulsed light signals to the left and right eyes and of pulsed sound signals to the left and right ears by simultaneously blanking out other visual and acoustical perceptions from the surroundings.

This device is to allow a self-treatment. Light-emitting diodes, which are separately controllable, are arranged in the left and right eyeglass lenses for the purpose of the optical stimulation. The light-emitting diodes are to be located horizontally and centrally on a darkened eyeglass lens, respectively, opaque surface which, in turn, is received by the eyeglass frame. Additionally, a cover enclosing the eyeglass frame is provided, which shields incident light on the side and thus confines the field of view of the user to the light-emitting diodes.

DE 10 2008 012 669 A1 discloses a device and a method for the visual stimulation, including a plurality of stimulation elements that generate visual stimulation signals which, when taken up by an eye of the patient and transmitted to a neuron population with a pathologically synchronous and oscillatory neuronal activity, reset the phase of the neuronal activity of the neurons. The control unit operates the stimulation unit such that at least two provided stimulation elements generate the visual stimulation signals with a time offset and/or with a different phase and/or different polarity.

In one of the embodiments according to DE 10 2008 012 669 A1 not only transmission glasses are explained, but also the arrangement of epicutaneous, i.e. attached to the skin of the patient, EEG electrodes. The control unit amplifies the potential difference measured by means of the EEG electrodes and uses said potential difference for actuating the transmission-modulated lenses of the transmission glasses after a corresponding combination.

The transmission glasses are formed of two rim parts, each with a transmission-modulated lens, ear pieces for mechanical attachment behind the ears of the patient, and a corresponding control unit altering the transmission of the transmission-modulated lenses of the glasses. Alternatively, reference is also made to the use of partially transparent or opaque light glasses as stimulation glasses.

The device for the electrostimulation of an eye according to DE 10 2010 027 201 A1 comprises a spectacles-like frame having a nose piece and, connected to the nose piece, an arrangement for attaching the frame to the patient's head.

At least one stimulation electrode is arranged on the nose piece. In addition, two electrode holders are provided on the nose piece between which an exchangeable wire-type stimulation electrode can be clamped.

Hence, according to this prior art, a mask-type structure is provided which lies on the patient's face, surface to surface, in the forehead region, cheek region and nose region, and has an adapted eye opening for the eye. The device in question allows by means of the electrodes a good, reproducible contact between the respective stimulation electrode and the eye to be treated, and is easily usable by a blind or nearly blind patient.

In addition, eyeglasses-like devices are known which include an eye-covering portion on which several electrodes are disposed. Specifically, devices are known, for instance according to US 2004/0176820, which have several electrodes provided around the eye area. The electrodes make punctual contact with the tissue directly surrounding the eyes and are connected to a stimulation apparatus that generates signals which are passed, as stimulation signals, by the electrodes into the eyes. A respective counter-electrode is fixed at an optional position of the patient's body.

The generic DE 10 2004 048 982 A1 shows an eye attachment for a stimulation device for stimulating the central nervous system and the brain waves of the human being. The eye attachment is designed to shield the eyes from external light incidence when worn in front of the eyes. Moreover, at least one light source is provided for emitting varying light signals, and the at least one light source is arranged on or integrated in the eye attachment such that the light thereof is not in direct contact with the eyes.

Also, at least one element illuminated by the at least one light source is integrated in or provided on the eye attachment such that the light from the light source is in indirect contact with the eyes via this illuminated element. Using the eyeglasses according to DE 10 2004 048 982 A1 does not involve any unpleasant high light intensities.

In one embodiment the illuminated element may have a flat configuration. The size of the surface and the arrangement thereof in the eye attachment, respectively, the integration thereof in the eye attachment may be coordinated in a such a manner that the field of view of one eye or both eyes is largely covered by the surface.

In one embodiment the light source is arranged on at least one lateral surface of a transparent plate connecting the front side to the rear side such that the light is emitted into the plate between the front side and the rear side. The light irradiated between the front side and the rear side of the plate is diffused in the plate and thus reaches the eye, respectively, eyes of the wearer of the eye attachment indirectly. Especially if the at least one light source is arranged on the upper lateral surface of the transparent plate an impression of a natural light incidence from above is created.

Based on the foregoing it is, therefore, the object of the invention to provide a further developed device for neurostimulation, comprising a mask, eyeglasses or similar eye attachment covering the eye area of the subject which is designed to be optically sealed from the surroundings and has an illuminated surface which is directed to the eye side of the subject and is connected to a source emitting light in the visible spectral range or contains such a light source, and having means for fixing it to the head of the subject, e.g. in the form of temples or an elastic band.

According to the invention, there is provided a base body, open on one side, e.g. made of a plastic material, which receives the respective illuminated surface and light source in its interior. To this effect, the base body has a cavity, not least in order to prevent a contact with the eyelids when wearing the eye attachment.

Electrode receptacles or electrode support surfaces are provided on the base body above and/or below the region covering the eye area.

Furthermore, a flexible, exchangeable cushion-like or mat-like surface body can be fixed to the base body, which has through openings that are complementary to the electrode receptacles or electrode support surfaces, wherein the surface body comes into contact with the forehead region of the eye area of the subject when the device is used, and the electrodes are designed to be epicutaneous.

The surface body can be easily separated from the base body after use and, especially in terms of hygiene, be disposed of or disinfected and reused depending on the type of surface material. The cushion-like or mat-like surface body causes a comfortable wearing sensation due to its material properties and prevents a lateral light incidence between the base body and the subject's skin.

In a preferred embodiment of the invention the electrodes are formed of two parts. Both electrode parts can be connected by a snap-in connection and be fixed to the surface body at the same time.

The electrodes can hence be separated from the surface body, so that a reuse of the electrodes is basically possible. The through openings in the surface body and the connection of the surface body to the base body guarantee a reproducible position of the electrodes any time.

In another embodiment of the invention it is possible to arrange a contact cap or a cap, respectively, corresponding cover absorbing moisture on the electrode part pointing to the eye area or forehead region.

Preferably, the contact cap may be made of a precious metal, e.g. gold, or have a coating of this type, so as to minimize the transition resistance between the electrode surface and the skin surface, and ensure a corresponding reproducibility on the resistance side.

The moisture-absorbing cap or the moisture-absorbing cover may be made of a foamed material, a felt material or a nonwoven material. The contact resistance can be minimized by targeted moistening, e.g. with a saline solution.

The above-described surface body may be made of a foam rubber material, a spacer fabric or a similar material.

The above-mentioned electrodes are arranged in a basically symmetrically distributed manner. In a preferred embodiment four electrodes are provided, wherein the electrodes are each located in the base body, respectively, surface body at the left and right eyes, above and below the eyes.

In one embodiment of the invention the illuminated surface may be configured as a diffusing disc, wherein radiation is coupled in via at least one of the respective front or side surfaces.

The diffusing disc(s) is (are) configured to be insertable and exchangeable on the base body from the open side.

The illuminated surface may also be a part of a miniature monitor, respectively, miniature display, however, so as to guarantee the desired optical excitation of the subject by the retina.

The base body is preferably made of an elastic material, or comprises a flexible, articulated section at least in the area lying on or above the nasal bone so as to ensure the wearing comfort and adequate contact on the head of the subject according to the anatomical conditions.

Moreover, electrical connection means, e.g. configured as plug or socket, in particular of a coaxial design, are provided on the base body.

In addition, at least one reference electrode or counter-electrode is arranged on the neck region or shoulder region of the subject.

The at least one reference electrode or counter-electrode can then be contacted with the corresponding connection means on the base body, wherein the device according to the invention is connected to a control unit supplying stimulation signals for the optical and electrical neurostimulation. The solution disclosed in DE 20 2006 021 009 may be used as such a stimulation device. The subject matter of this disclosure is herewith fully incorporated by reference.

The invention will be explained in more detail below by means of exemplary embodiments and schematic diagrams.

In the drawings:

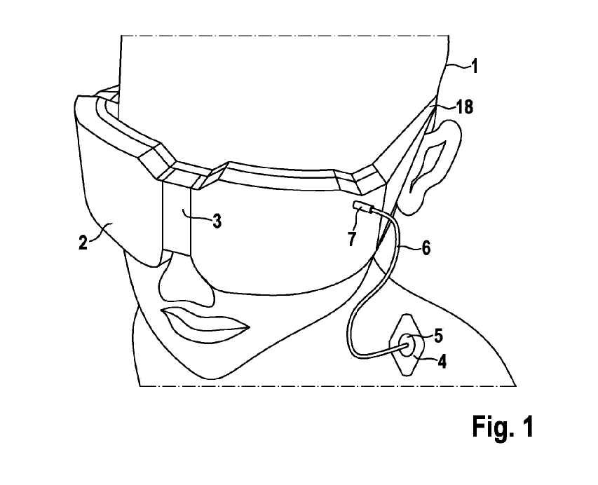

FIG. 1 shows a representation of a first embodiment of the device for neurostimulation according to the invention, configured as an eyeglasses-like mask, located on the head of a subject;

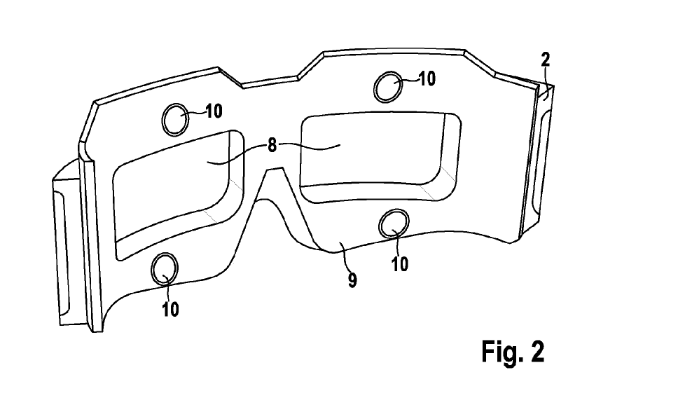

FIG. 2 shows a representation of the device according to the invention having a recognizable surface body, electrodes and illuminated surfaces, i.e. with a view into the inside of the base body, not including retaining pieces or a retaining band;

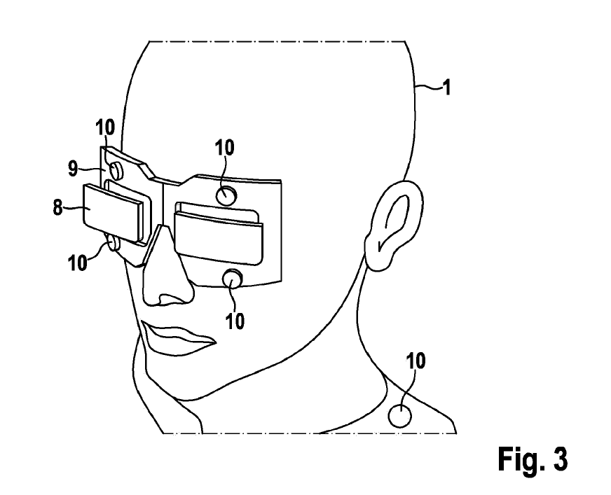

FIG. 3 shows a representation of the device without the base body, the side of the surface body facing away from the subject, with recognizable active electrodes in the eye area and forehead region, and the reference electrode or counter-electrode on the shoulder;

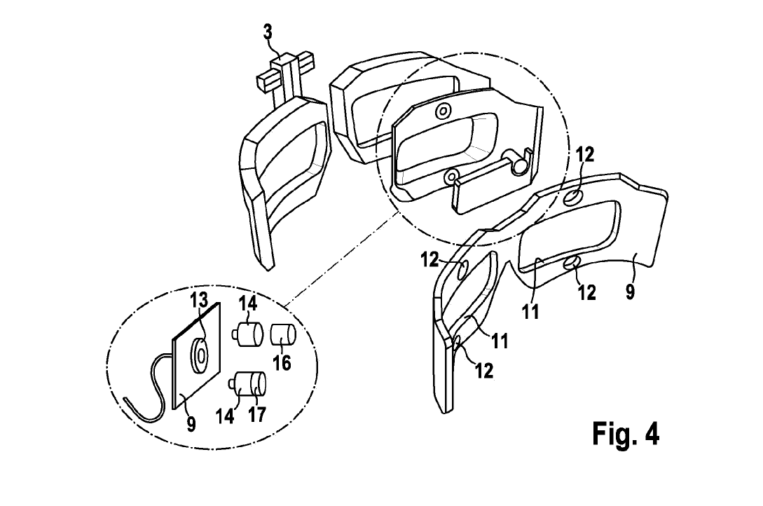

FIG. 4 shows an exploded view of essential components of the device according to the invention, with a detailed representation of the structure of the active electrodes located on the surface body; and

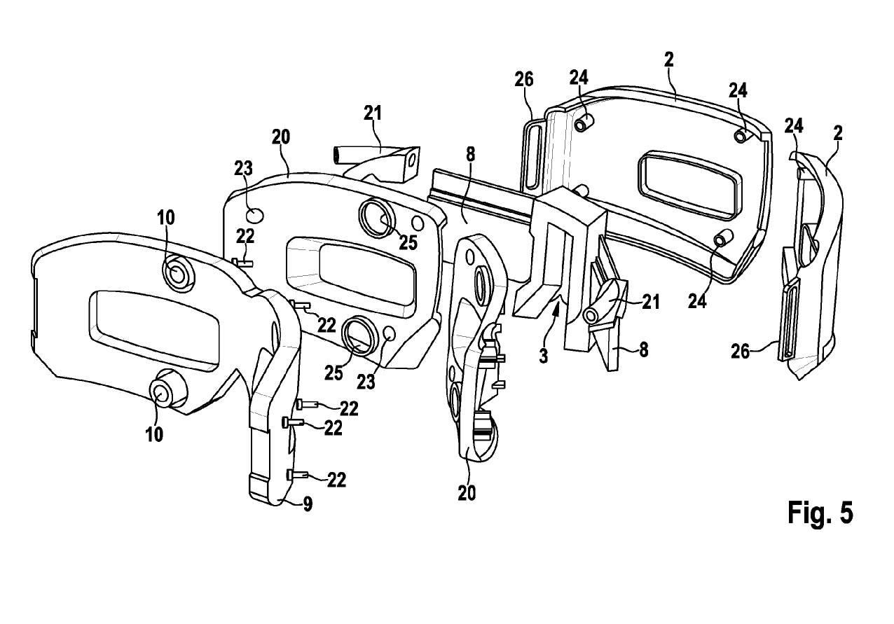

FIG. 5 shows an exploded view of a second embodiment of the device according to the invention.

According to FIG. 1 the device for neurostimulation according to the invention is fixed to the head of a subject 1.

FIG. 1 shows the base body 2, which is configured to create an eye attachment that opaquely covers the region of the eyes of the subject 1.

The base body 2 may be divided into two parts, namely a left-sided and right-sided eye cover with an elastic articulation 3 connecting the parts.

In the example shown, a reference electrode 4 is fixed in the left shoulder region of the subject 1, the reference electrode 4 comprising a contact 5 so as to establish, by cable 6, an electrical connection to another contact terminal 7 on the base body 2.

In the representation according to FIG. 2 the inside or rear side of the base body 2 is shown.

A light source for the left and right eyes, e.g. configured as illuminated surface 8, is located in the cavities of the base body 2. The illuminated surfaces 8 may also be realized as LED monitors or LCD monitors.

The side of the base body 2 pointing to the subject 1 is covered by a surface body 9, which is designed cushion-like or mat-like. The surface body 9 ensures a pleasant wearing sensation and can be easily replaced, and thus exchanged for hygienic reasons.

Electrodes 10, each arranged above and below the eye area, respectively, illuminated surfaces 8, project through openings in the surface body 9.

The epicutaneously configured electrodes 10 may have an external surface made of a precious metal, e.g. gold, so as to minimize the contact transition resistance to the subject's skin or make it reproducible, respectively.

For illustrating the arrangement of the surface body 9 with the electrodes 10 reference is made to FIG. 3 showing the device without the base body.

The exploded view and the detailed view of the structure of the electrodes according to FIG. 4 illustrates the sandwich structure of the device.

The surface body 9 comprises recesses 11 for an unobstructed view to the illuminated surfaces 8 and the aforementioned through openings 12 for the electrodes 10 to pass there through.

In a preferred embodiment the electrodes 10 are formed of two parts, namely a first part 13 and a second part 14.

The first part 13 and the second part 14 may be connected by a snap-in mechanism.

The electrode halves 13 and 14 may be fixed to the surface body 9 by interposing a corresponding section of the surface body 9.

To obtain reproducible properties it is further possible to provide the electrode half or electrode part 14, which is oriented in the direction of the skin surface of the subject 1, with a felt 16 or a precious metal contact surface 17.

The felt arrangement 16 which may be configured as a cap can be soaked with a liquid so as to reduce the contact transition resistance to the skin surface.

The electrodes, in particular electrode part 13, may be guided or fixed in an electrode receptacle on the base body 2 (the receptacle is not shown in the figures). Alternatively, a corresponding surface on the base body may also merely serve to support the respective electrode. The band 18 shown in FIG. 1 ensures a firm seat of the device according to the invention on the head of the subject 1.

Instead of the snap-in connection for the electrode parts or electrode halves 13, 14 as disclosed in FIG. 4 it is also possible to choose a screw connection or a bayonet-like fixing type.

The side of the surface body facing away from the skin of the subject may have an adhesion coating so as to ensure that the surface body is temporarily fixed to the base body.

If the surface body 9 is made, for example, of a spacer fabric it is possible to provide conductive pile threads so as to achieve an electrical contacting of the electrodes. Moreover, the spacer fabric absorbs moisture, i.e. sweat on the skin surface of the subject is absorbed and carried off, thereby further enhancing the wearing comfort of the device according to the invention.

In a respective embodiment of the surface body 9 electrode surfaces may be worked into same or be embedded in same in any other way. In this case, a contact connection to counter-contact elements on or in the base body then has to be provided merely on the rear side.

The surface body can be easily separated from the base body and, in terms of hygiene, be disposed of or, if a suitable material is used, be subjected to a disinfection step and reused.

In the embodiment of the device according to the invention of FIG. 5 the base body is formed by two outer shells 2 and two inner shells 20.

A carrier part with illuminated surfaces 8 is located between the outer shells 2 and the inner shells 20. The carrier part, which is formed of two halves in the example shown, is connected by an elastic nose piece 3. In addition, antikink elements 21 receiving the leads can be fixed to the carrier part with the illuminated surfaces.

An attachment is accomplished by screw connections 22 and through bores 23 in the inner shells and pin-shaped projections 24 on the outer shells 2, by simultaneously fixing the illuminated surfaces 8.

In the embodiment according to FIG. 5 the surface body 9 is configured as a foamed material pad and includes recesses for the electrodes 10. The electrodes 10 can be fixed to the inner shells and locking elements 25 provided there by means of a snap-in connection.

Lateral prolongations 26 are provided on the outer shells 2 which may serve to receive a retaining band, respectively, head band.

It will be appreciated that by using an electronic circuit, basically each of the electrodes may act permanently or temporarily as a reference electrode. A terminal 7 according to the first embodiment is required only if the reference electrode is to be fixed at a place outside the device according to the invention, e.g. in the shoulder region.

According to the invention it is basically possible to fix the device to the subject's head not only by means of a retaining band (see FIG. 1, reference number 18). Rather, the eyeglasses-like device may also be coupled to an EEG cap, wherein the EEG cap is tightened on the head, for example, by means of chin straps or shoulder straps.

Apart from the possibility to moisten the electrodes it is furthermore provided to configure the electrodes to comprise recesses into which a contact agent, e.g. an electrically conducting gel, can be introduced.

1. Device for neurostimulation via electrodes, comprising a mask, eyeglasses or other eye attachment covering an eye area of a subject which is configured to be optically sealed from the surroundings and has an illuminated surface which is directed to the eye side and is connected to a source emitting light in the visible spectral range or contains such a light source, the device being fixable to the head of the subject, and comprising:
a base body, open on one side, which receives the respective illuminated surface and light source in its interior, wherein electrode receptacles or electrode support surfaces are provided on the base body above and/or below the region covering the eye area, and
a flexible, exchangeable cushion-like or mat-like surface body fixable to the base body, the surface body having through openings that are complementary to the electrode receptacles or electrode support surfaces, wherein the surface body is configured to come into contact with the forehead region of the eye area of the subject when the device is used for epicutaneous application of the electrodes.

2. Device according to claim 1, wherein the electrodes are formed of two parts and both parts can be fixed and connected to the surface body by a snap-in connection.

3. Device according to claim 2, wherein a contact cap or a cap absorbing moisture is located on the electrode part pointing to the eye area or forehead region.

4. Device according to claim 3, wherein the contact cap is made of a precious metal or has a precious metal coating.

5. Device according to claim 3, wherein the moisture-absorbing cap is made of a foamed material, a felt material or a nonwoven material.

6. Device according to claim 1, wherein the surface body is made of a foam rubber material, a spacer fabric or a similar material.

7. Device according to claim 1, wherein the electrodes are arranged in a symmetrically distributed manner.

8. Device according to claim 1, wherein the illuminated surface is configured as a diffusing disc, wherein radiation is coupled in via at least one of respective front or side surfaces.

9. Device according to claim 8, wherein the diffusing Disc is configured to be insertable and exchangeable on the base body from the open side.

10. Device according to claim 1, wherein the illuminated surface is a miniature monitor or a miniature display.

11. Device according to claim 1, wherein the base body is made of an elastic material and/or an area lying on or above a nasal bone comprises a flexible, articulated section.

12. Device according to claim 1, wherein an electrical connector configured as plug or socket is provided on the base body.

13. Device according to claim 1, wherein at least one reference electrode or counter-electrode is provided in a neck-shoulder region of the subject.

14. Device according to claims 12, wherein at least one reference electrode or counter-electrode is provided in a neck-shoulder region of the subject, and is contacted with the electrical connector on the base body.

15. Device according to claim 2, wherein the electrodes include a recess-like formation serving to receive an electrically effective contact agent.

16. Device according to claim 1, wherein the base body is formed of several parts and has outer shells and inner shells, wherein illuminated surfaces are arranged between the outer shells and the inner shells.
